(19) United States
(12) Patent Application Publication
(10) Pub. No.: US 2024/0181155 A1
(43) Pub. Date: Jun. 6, 2024

(54) DEVICE FOR SUBCUTANEOUS DELIVERY OF A MEDICAMENT

(71) Applicant: Nuova Ompi S.r.l. Unipersonale, Padua (IT)

(72) Inventors: Damien Brasset, Milano (MI) (IT); Mattia Cattaneo, Limbiate (MB) (IT); Noemi Caloi, Verona (VR) (IT); Riva Christian, Cinisello Balsamo (MI) (IT)

(21) Appl. No.: 18/525,381

(22) Filed: Nov. 30, 2023

(30) Foreign Application Priority Data
Dec. 1, 2022 (IT) 102022000024792

(51) Int. Cl. A61M 5/142; A61M 5/172; A61M 5/32

(52) U.S. Cl. CPC A61M 5/14248; A61M 5/172; A61M 5/3216; A61M 5/3271; A61M 2205/33; A61M 2205/8206

(57) ABSTRACT

A device for subcutaneous delivery of a medicament includes a base surface facing the skin of a patient when the device is applied on the skin of the patient, and an injection needle to inject the medicament to the patient. The injection needle is movable between a rest position in which the injection needle is entirely arranged within the device and an injection position in which the injection needle protrudes at least partially from the device through a through opening formed in the base surface. The device includes a patch having an adhesive layer integrally formed with the base surface, and a removable protective layer. A cap is positioned at the base surface at the through opening and is integrated with the protective layer of the patch.

### CROSS REFERENCE TO RELATED APPLICATIONS

The present application claims priority to Italian Patent Application No. 102022000024792, filed Dec. 1, 2022, the disclosure of which is incorporated herein by reference in its entirety.

### TECHNICAL FIELD

The present disclosure relates to a device for subcutaneous delivery of a medicament. In particular, the present disclosure relates to a wearable type device configured to be applied on the body of a patient in order to allow the subcutaneous delivery of a predetermined dose of medicament.

### BACKGROUND

Wearable delivery devices can include medicament that is initially contained in a cartridge housed inside the device and is transferred to the body of the patient via a fluidic path. The cartridge can include a cylindrical container, made of a plastic or glass material, a plunger slidable inside the container to push the medicament out of the container and a pierceable septum that guarantees the sterility of the container until the beginning of the delivery of the therapy.

Typically, the fluidic path of wearable delivery devices includes a piercing needle to pierce the pierceable septum of the cartridge and a flexible tube that puts the piercing needle in fluid communication with an injection needle. The container of the cartridge is kept closed by the pierceable septum until the delivery of the medicament is required. When such a delivery is required, the piercing needle pierces the pierceable septum and opens the fluidic path, allowing the medicament to reach the patient by initially passing through the piercing needle, then through the flexible tube and finally through the injection needle, due to the thrust exerted by the plunger on the medicament inside the container.

### SUMMARY OF THE DISCLOSURE

The present disclosure relates to devices for subcutaneous delivery of a medicament, including a base surface, a patch, an injection needle, and a cap removably associated with the base surface.

The delivery of a medicament to a patient takes place after the insertion of the injection needle into the body of the patient. Such insertion occurs following activation of a movement mechanism configured to move the injection needle between a rest position in which the injection needle is entirely arranged inside the device and an injection position in which the injection needle protrudes from the device through a specifically provided through opening and penetrates the skin of the patient. The movement mechanism may also be configured to extract the injection needle from the skin of the patient at the end of the delivery of the therapy bringing it back inside the device.

WO 2015/187797A1 and WO 2021/252971A2, for example, describe devices provided with a cap applied on the device at the through opening and intended to be removed from the device before applying the device on the skin of the patient. In both WO 2015/187797A1 and WO 2021/252971A2 solutions intended to ensure that the cap is removed from the device only when the device must be actually used for the delivery of the therapy are provided. In particular, WO 2015/187797A1 discloses the use of sensors that signal a possible removal of the cap before the due time and of a controller that signals when the cap must be removed. WO 2021/252971A2 instead discloses the use of a locking mechanism that allows the removal of the cap only just before applying the device on the skin of the patient for the delivery of the therapy. The solutions disclosed in the devices of the abovementioned prior art documents are complex. It is an object of this disclosure to provide a constructively simpler solution adapted both to allow the cap to be kept on the device when the device is not used for the delivery of the therapy and to allow an easy removal of the cap when the device must actually be used for the delivery of the therapy, such as just before applying the device on the skin of the patient.

Many devices of the wearable type have a patch on the surface intended to face toward the skin of the patient when the device is in use. Such a patch comprises an adhesive layer covered by a protective layer. The latter is intended to be removed in order to expose the adhesive layer, so that the device can then be attached on the skin of the patient through the abovementioned adhesive layer. However, in some instances, a cap of the present disclosure can be incorporated with the protective layer of the patch.

In some aspects, the present disclosure therefore relates to a device for subcutaneous delivery of a medicament, comprising:
a base surface intended to face toward the skin of a patient when the device is applied on the skin of the patient and comprising a through opening;
a patch comprising an adhesive layer and a protective layer, wherein the adhesive layer is integrally associated with the base surface and is intended to be attached to the skin of the patient upon removal of the protective layer;
an injection needle configured to inject the medicament to a patient when the device is applied on the skin of the patient, the injection needle being movable between a rest position in which the injection needle is entirely arranged within the device and an injection position in which the injection needle protrudes at least partially from the device through the through opening;
cap removably associated with the base surface at the through opening; wherein the cap is integrally associated with the protective layer.

These, and other aspects, can include one or more of the following features. The cap can comprise a collar integrally associated with a portion of the protective layer by interposition of a double-sided adhesive element. The cap can be made of a rigid material. The device can comprise a shielding element arranged around the through opening and movable between a first operating position in which the shielding element protrudes from the device and a second operating position in which the shielding element does not protrude from the device, and an elastic element configured to exert a pushing action on the shielding element, the pushing action being suitable to keep the shielding element in the first operating position before removing the protective layer, where the cap is configured to house the shielding element before the protective layer is removed. The cap can be configured to house with clearance the shielding element. The device can further comprise a flexible sleeve connected to the device around the through opening and the shielding element to form a water-tight seal, the flexible sleeve being integrally associated with the shielding element. The flexible sleeve can be defined by a bellows-type membrane, and the membrane and the cap can have a truncated conical shape. The shielding element can include a base provided with a through hole configured to allow the passage of the injection needle during the movement of the injection needle between the rest position and the injection position, and the flexible sleeve can be integrally associated with the base. The flexible sleeve can comprise a base surface integrally associated with the base of the shielding element, and the base surface can cover the through hole and be pierceable by the injection needle when the injection needle moves from the rest position to the injection position. The device can further comprise a position sensor configured to detect a contact of the device with the skin of the patient, the position sensor comprising a magnet integrally connected to the shielding element and a Hall effect sensor arranged in the device close to the magnet. The magnet can be integrally connected to the base.

In the device of the present disclosure, it is possible to remove the cap only by removing the protective layer of the patch. Since the latter is removed just before applying the device on the skin of the patient for the delivery of the therapy, the cap remains applied on the device until when the device must be actually used.

Furthermore, the operation of preparing the device for use can be simple. For example, with a single action, the patient can remove the protective layer of the patch and the cap to expose the adhesive layer of the patch.

Certain features of the device of the present disclosure are described herein. Each of these features can be provided individually or in combination with the others.

In some embodiments, the cap comprises a collar integrally associated with a portion of the protective layer by interposition of a double-sided adhesive element. The latter allows the cap to be firmly bound to the protective layer, making it de facto integral to the latter.

In some embodiments, the cap is made of a rigid material.

In some embodiments, the device further comprises a shielding element arranged around the through opening and movable between a first operating position in which the shielding element protrudes from the device and a second operating position in which the shielding element does not protrude from the device. This shielding element allows to protect the patient from accidental contacts with the tip of the injection needle when the device is not applied on the skin of the patient, if the injection needle is outside the device.

In some embodiments, it is provided that the shielding element is in the first operating position before applying the device on the skin of the patient and surrounds the tip of the injection needle in case the latter for some reason is outside the device. In fact, although it is provided that before removing the device from the skin of the patient the injection needle is returned into the device, it cannot be ruled out that following an accidental or voluntary removal of the device from the skin of the patient the injection needle is outside the device, with the risk for the patient of getting stung.

In some embodiments, the device further comprises an elastic element associated with the shielding element and configured to exert on the shielding element a pushing action which is suitable to keep the shielding element in the first operating position before removing the protective layer. Thus, in its initial configuration, the shielding element protrudes from the device and is free to move from outside to inside the device.

In some embodiments, the cap is configured to house the shielding element, for example, with clearance, before removing the protective layer. The cap therefore prevents access to the shielding element when the device is not used for the delivery of the therapy, i.e. before applying the device on the skin of the patient.

In some embodiments, the device comprises a flexible sleeve associated water-tightly to the device around the through opening and integrally associated with the shielding element.

Being integrally associated with the shielding element, the flexible sleeve moves with the shielding element between the abovementioned first operating position in which the flexible sleeve and the shielding element extend outside the device and the abovementioned second operating position in which the flexible sleeve and the shielding element are housed inside the device. Therefore, by associating the flexible sleeve with the device around the shielding element so as to be watertight, it is ensured the impermeability of the device and the seal also against powders at the through opening through which the injection needle exits for the delivery of the therapy.

In some embodiments, the shielding element comprises a base provided with a through hole to allow the passage of the injection needle during the movement of the injection needle between the rest position and the injection position.

In some embodiments, the base comprises a raised edge defining in the shielding element a recess that houses the tip of the injection needle both when the shielding element is in the second operating position and the injection needle is in the rest position and when the shielding element is in the first operating position and the injection needle is in the injection position. The raised edge surrounds the tip of the injection needle and prevents the user from coming into contact with the tip if the injection needle is outside the device and the device is not applied on the skin of the patient.

In some embodiments, the flexible sleeve is integrally associated with the base.

In some embodiments, the flexible sleeve is defined by a membrane having an inner surface integrally associated with the outer surface of the base.

In some embodiments, the membrane is shaped like a bellow.

In some embodiments, the membrane has a substantially conical or truncated conical shape. In this way the membrane can be telescopically folded inside the device when the device is applied on the skin of the patient, thus occupying an extremely limited space, to the benefit of the compactness of the device.

In some embodiments, the membrane and the cap have a truncated conical shape.

In some embodiments, the flexible sleeve has a base surface integrally associated with the base of the shielding element.

In some embodiments, the base surface covers the through hole of the base of the shielding element and is pierceable by the injection needle when the injection needle moves from the rest position to the injection position. In this case the flexible sleeve covers with continuity the shielding element also covering the through hole of the base of the shielding element and it is the injection needle itself that pierces the base surface of the flexible sleeve when it comes out from the shielding element and, therefore, from the flexible sleeve.

In certain embodiments, the device further comprises a position sensor configured to detect a contact of the device with the skin of the patient. The abovementioned sensor ensures that the device cannot be put into operation before being applied on the body of the patient or, in other words, the device can be put into operation only when it is effectively in the condition of delivering the medicament. This is particularly advantageous since the medicaments used in these devices are typically very expensive and that in order to ensure the safety of the patients, the device is considered to have been used immediately after it is put into operation, regardless of whether the medicament is then actually delivered to the patient or whether such a delivery is successful.

In some embodiments, the position sensor comprises a magnet integrally associated with the shielding element and a Hall effect sensor arranged in the device close to the magnet. In this way, the detection of the contact between the device and the body of the patient takes place due to the movement of the shielding element from outside to inside the device as soon as the device is applied on the body of the patient. In fact, this movement causes the magnet to approach the Hall effect sensor. The housing of the shielding element inside the cap becomes in this case particularly advantageous due to the fact that it prevents the patient from voluntarily or accidentally moving the shielding element when the device is not applied on the skin of the patient, thus limiting the risks of putting the device into operation before it is applied on the body of the patient.

In some embodiments, the magnet is integrally associated with the base.

### BRIEF DESCRIPTION OF THE DRAWINGS

Further features and advantages of the present disclosure will become clearer from the following detailed description of preferred embodiments thereof, made with reference to the accompanying drawings and given for indicative and non-limiting purpose. In such drawings:

FIG. 1 is a perspective top view of an example device according to the present disclosure without a patch;

FIG. 2 is a perspective bottom view of the device of FIG. 1 provided with a patch;

FIG. 3 is a perspective bottom view of the device of FIG. 1 after removing the protective layer of the patch;

FIG. 4 is a perspective sectional view of the device of FIG. 1 before being applied on the skin of the patient;

FIG. 5 is a perspective sectional view of the device of FIG. 1 when applied on the skin of the patient.

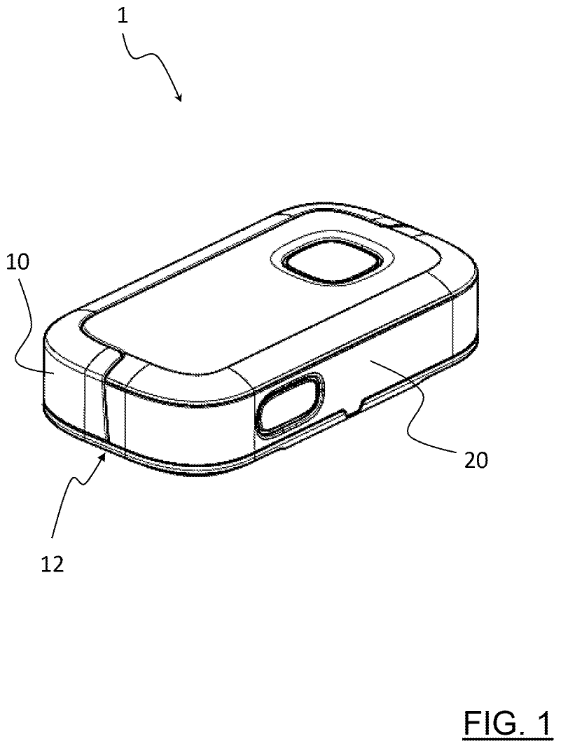

### DETAILED DESCRIPTION

In the accompanying Figures, an example device in accordance with the present disclosure is indicated with 1. The application of the device 1 on the body of the patient is carried out by a user, for example a doctor or a nurse or the patient.

The example device 1 comprises a delivery module 10 comprising a base surface 12 intended to be applied on the skin of a patient and a control module 20 intended to control the operation of the device 1. In particular, the control module 20 comprises a control unit (not visible), usually made in a printed circuit and configured to control the delivery of the medicament from the delivery module 10, and a power supply battery (also not visible) configured to power the control unit.

The device 1 is obtained by mutually coupling the delivery module 10 and the control module 20. Such a coupling is reversible, i.e. the two modules 10 and 20 can be decoupled after having been coupled.

One or more gaskets are interposed between the delivery module 10 and the control module 20 in order to ensure the tightness of the mutual coupling.

In some instances, the delivery module 10 is disposable, while the control module 20 is reusable. In other words, the delivery module 10 is configured to be applied on the body of the patient only once and for a certain period of time in order to deliver the medicament, completely or in part, in one or more subsequent injections, even temporally spaced apart. Conversely, the control module 20 can be used several times, by coupling it from time to time with a new delivery module.

A cartridge is housed in the delivery module 10. The cartridge comprises a substantially cylindrical container, made of a plastic or glass material, containing a medicament to be delivered to a patient and a pierceable septum that closes the container at one end thereof and guarantees its sterility until the device 1 is used.

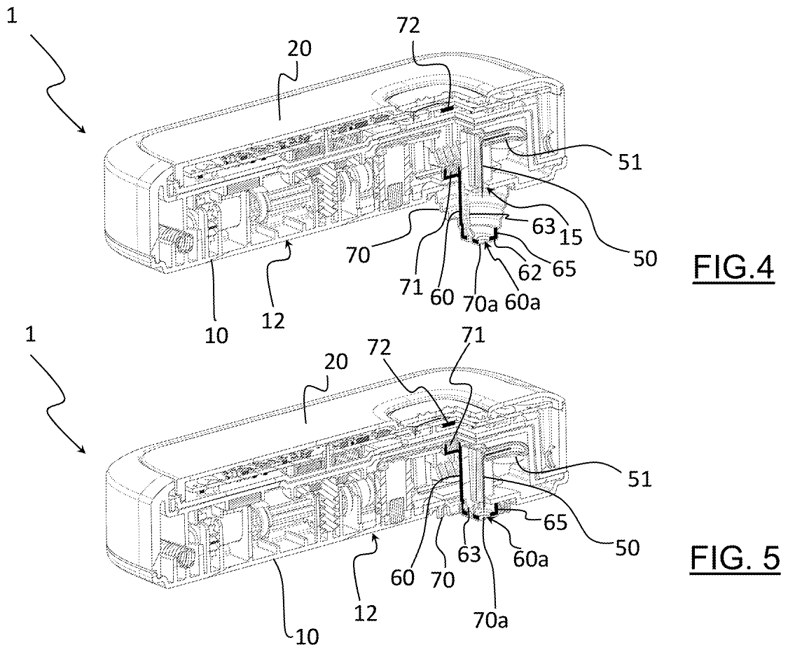

The delivery module 10 further comprises a fluidic path configured to be travelled by the medicament during the subcutaneous delivery of the medicament, allowing the passage of the medicament from the cartridge to the body of the patient. This fluidic path comprises a piercing needle configured to pierce the septum of the cartridge, an injection needle 50 (FIGS. 4 and 5) configured to be inserted into the body of the patient and a flexible tube 51 that puts the abovementioned needles in fluid communication.

In the present description and in the subsequent claims, the term “fluidic path” is used to indicate any element or assembly of elements that is configured to be connected, at a first end thereof, to the cartridge housed inside the delivery device and comprising, at an end thereof opposite to the abovementioned first end, an injection needle intended to be inserted into the body of the patient to allow the transfer of the medicament from the cartridge to the body of the patient.

The injection needle 50 is movable through the through opening 15 between a rest position in which it is entirely arranged inside the device 1, as shown in FIGS. 4 and 5, and an injection position in which it protrudes from the device 1 through the through opening 15.

As shown in FIGS. 3-5, a shielding element 60, depicted in bold in FIGS. 4 and 5, is arranged around the opening 15 of the base surface 12.

The shielding element 60 is movable between a first operating position in which the shielding element is in an extended configuration and protrudes from the delivery module 10 (FIGS. 3 and 4), possibly arranging itself around the injection needle 50 if the latter is outside the delivery module 10, and a second operating position in which the shielding element 60 is in a retracted configuration and is arranged entirely inside the delivery module 10 at the base surface 12 (FIG. 5).

The shielding element 60 comprises a base 60a which, when the shielding element 60 is in its first operating position, is located distal from the base surface 12.

In the base 60a there is formed a through hole 62 adapted to allow the passage of the injection needle 50 when the device 1 is applied on the skin of the patient (and therefore when the shielding element 60 is in the retracted configuration).

As shown in FIGS. 4 and 5, the base 60a comprises a raised edge 65 which defines in the shielding element 60 a recess for housing the tip of the injection needle 50 both when the shielding element 60 is in the second operating position and the injection needle 50 is in the rest position (FIG. 5) and if the injection needle 50 is in the injection position and the shielding element 60 in its first operating position.

Before applying the device 1, the shielding element 60 is kept in its first operating position by the pushing action exerted by an elastic element 63 which, in the embodiment illustrated herein, is a compression spring.

As shown in FIGS. 4 and 5, the device 1 comprises a position sensor configured to signal to the control unit that the device 1 has been positioned on the skin of the patient, so as to proceed with the delivery of the therapy.

In particular, the position sensor comprises a magnet 71 integrally associated with the base 60a of the shielding element 60 and a Hall effect sensor 72 integrally associated with the control module 20 close to the magnet 71. When the device 1 is applied on the skin of the patient, the shielding element 60 moves inside the device 1, its base 60a moves towards the base surface 12 of the delivery module 10 and the magnet 71 approaches the Hall effect sensor 72.

As shown in FIGS. 3-5, the device 1 further comprises a flexible sleeve 70 associated water-tightly with the base surface 12 of the delivery module 10 around the through opening 15.

The flexible sleeve 70 surrounds the shielding element 60 and comprises a base surface 70a integrally associated with the base 60a of the shielding element 60.

In the embodiment illustrated herein, the flexible sleeve 70 is defined by a bellows-type membrane having a truncated conical shape.

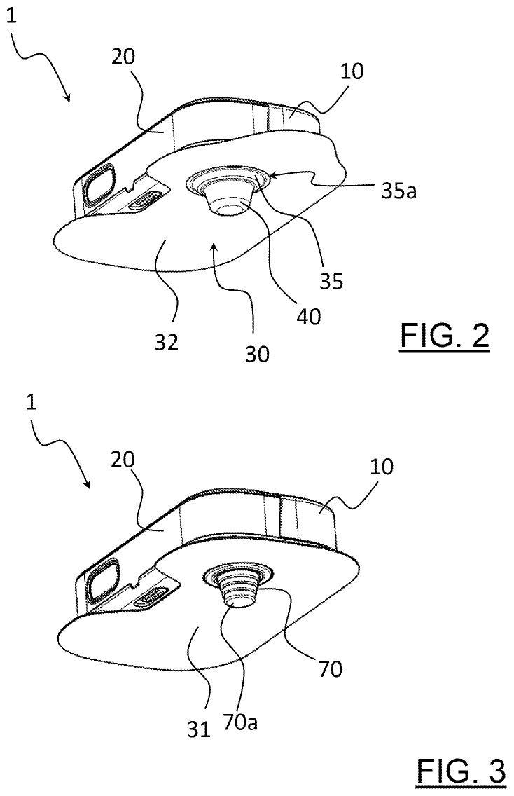

As shown in FIG. 2, the cap 40 also has a truncated conical shape and houses with clearance the shielding element 60 and the flexible sleeve 70.

The base surface 70a of the flexible sleeve 70 covers the through hole 62 of the shielding element 60 and is pierceable by the injection needle 50 when the injection needle 50 moves from the rest position to the injection position.

As shown in FIGS. 2 and 3, a patch 30 is applied on the base surface 12 of the delivery module 10.

The patch 30 comprises an adhesive layer 31 integrally attached to the base surface 12 and a protective layer 32 removably attached to the adhesive layer 31. Before performing the delivery of the therapy, the protective layer 32 is removed and the device 1 is attached to the skin of the patient through the adhesive layer 31.

The device 1 further comprises a cap 40 integrally attached to the protective layer 32. In particular, the cap 40 comprises a collar 35 attached to the protective layer 32 by interposition of a double-sided adhesive element 35a.

The cap 40 is made of a rigid material and is shaped so as to completely house with clearance the shielding element 60 and the flexible sleeve 70 that surrounds the shielding element 60 when the latter is in its first operating position.

In the embodiment illustrated herein, the cap 40 has a truncated conical shape.

As already mentioned, at first the shielding element 60 is in its extended configuration of FIGS. 3 and 4 and the cap 40 covers the shielding element 60, as shown in FIG. 2.

Before applying the device 1 on the skin of the patient, the patient removes the protective layer 32 of the patch 30, removing simultaneously therewith also the cap 40 and thus releasing the shielding element 60 and the adhesive layer 31 of the patch 30.

The patient can then proceed to apply the device 1 on his/her skin by attaching the device through the adhesive layer 31 of the patch 30. The skin of the patient counteracts the pushing action exerted by the elastic element 63 and makes the shielding element 60 retract inside the delivery module 10, bringing the magnet 71 closer to the Hall effect sensor 72 and thus allowing the control unit to establish that the device 1 is in contact with the skin of the patient and to activate the delivery of the therapy.

1. A device for subcutaneous delivery of a medicament, comprising:
a base surface configured to face toward the skin of a patient when the device is applied on the skin of the patient, the base surface comprising a through opening;
a patch comprising an adhesive layer and a protective layer, wherein the adhesive layer is integrally associated with the base surface and is configured to be attached to the skin of the patient upon removal of the protective layer;
an injection needle configured to inject the medicament to the patient when the device is applied on the skin of the patient, the injection needle being movable between a rest position in which the injection needle is entirely arranged within the device and an injection position in which the injection needle protrudes at least partially from the device through the through opening; and
a cap removably associated with the base surface at the through opening;
wherein the cap is integrally associated with the protective layer.

2. The device of claim 1, wherein the cap comprises a collar integrally associated with a portion of the protective layer by interposition of a double-sided adhesive element.

3. The device of claim 1, wherein the cap is made of a rigid material.

4. The device of claim 1, comprising:
a shielding element arranged around the through opening and movable between a first operating position in which the shielding element protrudes from the device and a second operating position in which the shielding element does not protrude from the device; and
an elastic element configured to exert a force on the shielding element, the force being suitable to keep the shielding element in the first operating position before removing the protective layer;
wherein the cap is configured to house the shielding element before the protective layer is removed.

5. The device of claim 4, wherein the cap is configured to house the shielding element with clearance.

6. The device of claim 4, further comprising a flexible sleeve connected to the device around the through opening and the shielding element to form a water-tight seal, the flexible sleeve being integrally associated with the shielding element.

7. The device of claim 6, wherein the flexible sleeve is defined by a bellows-type membrane, and the membrane and the cap have a truncated conical shape.

8. The device of claim 6, wherein the shielding element comprises a base provided with a through hole configured to allow passage of the injection needle during movement of the injection needle between the rest position and the injection position, and the flexible sleeve is integrally associated with the base.

9. The device of claim 8, wherein the flexible sleeve comprises a base surface integrally associated with the base of the shielding element, and the base surface covers the through hole and is pierceable by the injection needle when the injection needle moves from the rest position to the injection position.

10. The device of claim 8, further comprising a position sensor configured to detect a contact of the device with the skin of the patient, the position sensor comprising a magnet integrally connected to the shielding element and a Hall effect sensor arranged in the device proximate the magnet.

11. The device of claim 10, wherein the magnet is integrally connected to the base.

12. The device of claim 9, further comprising a position sensor configured to detect a contact of the device with the skin of the patient, the position sensor comprising a magnet integrally connected to the shielding element and a Hall effect sensor arranged in the device proximate the magnet.

13. The device of claim 4, further comprising a position sensor configured to detect a contact of the device with the skin of the patient, the position sensor comprising a magnet integrally connected to the shielding element and a Hall effect sensor arranged in the device close to the magnet.

14. The device of claim 7, wherein the shielding element comprises a base provided with a through hole configured to allow passage of the injection needle during movement of the injection needle between the rest position and the injection position, and the flexible sleeve is integrally associated with the base.

15. The device of claim 14, wherein the flexible sleeve comprises a base surface integrally associated with the base of the shielding element, and the base surface covers the through hole and is pierceable by the injection needle when the injection needle moves from the rest position to the injection position.

16. The device of claim 5, further comprising a flexible sleeve connected to the device around the through opening and the shielding element to form a water-tight seal, the flexible sleeve being integrally associated with the shielding element.

17. The device of claim 16, wherein the flexible sleeve is defined by a bellows-type membrane, and the membrane and the cap have a truncated conical shape.

18. The device of claim 2, wherein the cap is made of a rigid material.

19. The device of claim 18, comprising:
a shielding element arranged around the through opening and movable between a first operating position in which the shielding element protrudes from the device and a second operating position in which the shielding element does not protrude from the device; and
an elastic element configured to exert a force on the shielding element, the force being suitable to keep the shielding element in the first operating position before removing the protective layer;
wherein the cap is configured to house the shielding element before the protective layer is removed.

20. The device of claim 2, comprising:
a shielding element arranged around the through opening and movable between a first operating position in which the shielding element protrudes from the device and a second operating position in which the shielding element does not protrude from the device; and
an elastic element configured to exert a force on the shielding element, the force being suitable to keep the shielding element in the first operating position before removing the protective layer;
wherein the cap is configured to house the shielding element before the protective layer is removed.
